(19) United States
(12) Patent Application Publication
(10) Pub. No.: US 2004/0129275 A1
(43) Pub. Date: Jul. 8, 2004

(54) Treatment bed, treatment apparatus and treatment device

(76) Inventor: Richard Markoll, Munchen (DE)

Correspondence Address: FITCH EVEN TABIN AND FLANNERY, 120 SOUTH LA SALLE STREET, SUITE 1600, CHICAGO, IL, 60603-3406, US

(21) Appl. No.: 10/469,286

(22) PCT Filed: Feb. 28, 2002
(86) PCT No.: PCT/EP02/02184

(30) Foreign Application Priority Data
Mar. 1, 2001 (DE) 101 09 849.9

(51) Int. Cl. A61G 15/00

(52) U.S. Cl. 128/845

(57) ABSTRACT

The invention relates to a treatment device (10) comprising a treatment bed (20) and treatment apparatus (50). The treatment bed (20) consists of a frame (22) and at least one bearing element (33) which can be placed on the frame. At least one elongate recess (29,34) extending in the longitudinal direction of the frame is provided in the frame (22) and the bearing element (33), extending from the head section of the treatment bed in a centre area in the direction of the width thereof. The treatment area (50) comprises a bearing part (54) for the head of a patient, said bearing part essentially taking the shape of a ring segment. A cover part (58) which can be placed on at least one free end of the bearing part by means of a pivoting device is also provided. The at least one cover part is connected to the bearing part by means of a signal transmission device (41). The treatment apparatus (50) is completed by means of a fixing device (52) for the treatment device and a signal generating device and/or control device (42) connected to the treatment apparatus.

### FIELD OF THE INVENTION

The present invention relates to a treatment bed, a treatment apparatus and a treatment device which are suitable for treating the head area of patients.

It is possible to successfully treat patients with, for example, tinnitus complaints by means of radiation therapy, for instance by means of radiation with specific signals such as pulsating fields which are applied via a magnetic field. To do so, the patient and in particular the area of their head with the complaint is subjected to radiation treatment over a relatively long period of time of approximately one hour. This means that the patient is sitting or lying on a treatment device for a predetermined period of time, whilst the signals, for example the specific signals such as pulsating fields applied via a magnetic field, are applied to his head in a suitable manner.

So that the rays can be suitably and effectively applied to the head area of the patient, a treatment bed in conjunction with the treatment apparatus must furthermore ensure that the patient does not significantly alter his treatment position during the treatment.

### PRIOR ART

Treatment beds having various adjustment mechanisms for different treatments are known from the prior art. Massage beds, for example, are known which have a head-shaped recess in the area in which the patient rests his head so that a patient lying on his stomach can breathe and lie comfortably throughout the duration of the massage treatment without turning his head and thus generating tension.

Correspondingly shaped beds are also known for other kinds of treatments and examinations.

Various treatment wires are also known for treating the head area. Known, for example, are pad-shaped bearing elements, headphone-like treatment devices or caps for radiation therapy, also with magnetic fields. Tube-shaped elements having a fixed diameter of the receiving opening for the patient's head are also used.

### DESCRIPTION OF THE INVENTION

The object of the invention is to propose a treatment bed, a treatment apparatus and a treatment device which, during a longer treatment of the head area of a patient, in particular by means of radiation therapy, allow the patient to rest in a comfortable, defined position on the bed and as regards the treatment apparatus, with, at the same time, the treatment bed and the treatment apparatus and treatment device remaining adaptable to the different body measurements of a patient.

This object is solved by means of a treatment bed having the features of claim 1, a treatment apparatus having the features of claim 17 and a treatment device having the features of claim 23.

As regards the treatment bed, the invention is based on the idea that the treatment bed is suitable, owing to its elongated recess which extends in the longitudinal direction of the treatment bed, for accommodating a treatment apparatus which can be adjusted to the length of a patient's body by means of a suitable device which is shiftable within the elongated recess, so that the treatment apparatus is suitably arranged in the head area of the patient. The elongated recess, several of which can also be provided if the treatment apparatus is to be fixed by several supports, is thereby arranged in such a way that it extends so far beyond the head section of the treatment bed that both patients requiring the entire length of the treatment bed owing to their height as well as comparatively small patients, for whom the apparatus is adjusted by means of the elongated recesses at the end of the recesses which is opposite to the head section, can be specifically treated.

As regards the treatment apparatus, the invention is based on the idea of designing said treatment apparatus so that it can be adapted to the different head circumferences of patients. Since it is sensible, for example for radiation treatment, to surround the head in a circular manner by means of a treatment device, at least one pivotably arranged cover part is attached in addition to a bearing part on which the patient's head is placed during the treatment. Thus, when the cover part is open, the patient's head can be placed in the treatment apparatus in the axial direction of the cylindrical opening (in the closed state) of the treatment apparatus. It is also possible to naturally place the head in said treatment apparatus from above. It is nevertheless ensured that the patient's head is surrounded in the closed state by the bearing part and at least one cover part so that an effective treatment is possible.

Finally, the treatment device according to the invention is based on the idea of integrating the adjustment mechanism, and thus the adapting mechanism, of the treatment bed and the treatment apparatus in a treatment device which is easily adaptable to the needs of a patient, in particular as regards his height and the circumference of his head. Owing to the design of the treatment device according to the invention, a patient to be treated can sit or lie on the treatment bed in a normal manner and can place his head in the treatment apparatus by lowering his upper body. No complicated sliding or the like is necessary.

Advantageous embodiments are described by the remaining claims.

The treatment bed therefore advantageously comprises an inclinable head section. The patient can thereby be treated in a comfortable sitting position which corresponds to an ergonomic resting position. The head section can be unchangeably inclined, that is to say the treatment bed has no position in which the patient actually lies, or the inclination of the head section can also be optionally adjusted by means of a suitable inclining mechanism and can be adjusted, as necessary, to different inclinations so that a comfortable treatment position for the patient can be selected. This is particularly advantageous since the patient has to spend a relatively long period of time on the bed during each treatment, i.e. approximately one hour. It must furthermore be taken into consideration that several treatments normally have to be carried out at predetermined intervals, and thus a position which is comfortable for the patient during treatment clearly contributes to the well-being of the patient.

The inclinable head section thereby advantageously takes up approximately half of the length of the treatment bed. This division is preferred since in this case, the buttocks and legs of the patient can be arranged in a basically horizontal position whereas the torso and head area can be supported by the treatment bed in an inclined position.

A simple adjustment mechanism for the treatment bed, in particular for inclining the head section of the treatment bed, can be obtained by means of a device as known, for example, in deckchairs. The supporting body, which is advantageously provided with the legs of the frame of the treatment bed, which stand on the floor, comprises at least one engaging area for a supporting part which is attached in a pivotable manner to the head section. By engaging the supporting part in the engaging area, the head part is brought into the inclined position and is supported in this inclined position by the supporting part. By providing several engaging areas on the supporting body, which could, for example, be designed in stages, an adjustment to several inclined positions as compared to the basically horizontal foot section of the frame is made possible. In this description, “longitudinal direction” consistently means the main direction of extension of the treatment bed, which corresponds to the head/foot direction of a patient when he is resting on the treatment bed. “Head section” is to be understood as the area of the treatment bed in which a patient rests his head during the treatment and accordingly, “foot section” is to be understood as the area of the treatment bed in which the feet of the patient are located during the treatment.

The frame of the treatment bed can preferably be made of different materials, for instance wood or metal. Wood offers the advantage of an appealing appearance whereas metal has proven successful in medical practices as well as in clinics/hospitals for reasons of hygiene. Metal is furthermore characterised by a high robustness.

According to an advantageous embodiment, the bearing element is a softly padded bearing element, for example by means of a foam inlay, which is provided with a corresponding cover. This helps, in particular, the comfort of the patient. Furthermore, the soft padding can cause an anatomic shaping of the bearing element, so that during the treatment, the areas of the patient's body are optimally supported by the treatment bed and in particular the bearing element thereof. This again leads to the patient feeling that he is lying or seated comfortably. The design of the bearing element by means of an inner padding and a corresponding cover is also advantageous since if the cover gets dirty, it can be simply cleaned separately from the padding.

The bearing element advantageously comprises a separate head part and a separate foot part. By designing the bearing element in at least two parts, it is ensured that in the case of an inclinable frame, no bulges which are uncomfortable for the patient occur in the bearing element in the area in which the frame bends. The bearing part can, of course, also be comprised of more than two parts. In particular, the head part and/or the foot part, for example, could for its part be designed in multiple parts.

An ergonomic design is furthermore preferred for the treatment bed. This is functionally achieved by the bearing element. In the case of the aforementioned padded bearing element, the ergonomic design of the treatment bed and, in particular, of the bearing element, can be simply achieved by means of a corresponding design of the padding and the cover. However, an ergonomic design is also possible for other bearing elements, for example a taut material between the frame, a correspondingly shaped synthetic material, correspondingly shaped wood or the like. It is particularly advantageous if, in the area in which the buttocks of the patient rest, the bearing element has a depression as compared to the area in which the legs of the patient rest. This results on the one hand in the legs of the patient lying in a slightly elevated position during the treatment as compared to the buttocks, which is advantageous for the blood circulation and thus in turn contributes to the well-being of the patient, and, on the other hand, a predetermined position of the patient on the treatment bed is achieved by means of the depression. This is practical since it thereby prevents the patient from significantly altering his position during the treatment and thus prevents, for example, radiation using a radiation device positioned in advance from acting on the wrong body parts. Furthermore, this design also allows a reduced control effort by personal since the probability of the patient changing his position is reduced.

The area supporting the lumbar region is also preferably designed in a raised manner as compared to the area in which the treatment apparatus is placed. This serves, in particular, to maintain the correct treatment position since if a patient undesirably changes his position, he immediately notices that his position on the treatment bed is no longer correct since he feels uncomfortable.

For aesthetic reasons, it is preferred for the bearing element in the area of the at least one recess in the frame to have a recess which exposes the recess present in the frame. A non-blocking and simple operation of the adjustment mechanism of the treatment apparatus is thereby ensured. For aesthetic reasons, it is preferred for this recess in the bearing element to be coverable by means of one or more filling pieces. This is not absolutely necessary as regards the functionality, but it does, however, offer a more appealing appearance if there is the impression that there are no gaps in the bearing element.

An adjustment mechanism for adjusting a treatment apparatus for the head area of a patient is preferably provided. The adjustment mechanism operates in such a way that it can shift the treatment apparatus in the longitudinal direction, i.e. in the head/foot direction of the patient.

In an inexpensive embodiment, the adjustment mechanism advantageously comprises a plate fixed to the side of the frame facing away from bearing element side. Fixing can be carried out, for example, by means of screws. Alternative fixing means, for example, an adhesive joining or the like, are also conceivable. At least one support for the treatment apparatus is shiftably attached to this plate, which advantageously reaches through the corresponding recesses in the frame and the bearing element which normally lies on the frame. This furthermore results in the elongated recesses in the frame being shaped in a preferred embodiment in such a way that they let through the support or supports for the treatment apparatus and allow the shifting of the support to occur without friction. An excessive width of the excesses is not preferred since in this case, the stability and rigidity of the frame would be reduced.

According to a preferred embodiment, the at least one support is shiftable by means of a spindle and a crank for operating the spindle. This is a reliable shifting mechanism which is easy to operate and can be easily adjusted to exactly the height of the patient.

According to a preferred embodiment, the treatment bed furthermore comprises a receptacle for a control device and/or a signal generating device for a treatment apparatus. Such a receptacle, which can be attached, for example in the form of an open cuboid, to the treatment bed, for instance in the foot section thereof, offers a compact accommodation of the control device and signal generating device on the treatment bed so that an additional frame for receiving this device is not necessary. This again contributes to an appealing appearance of the entire device. The receptacle for the control device and/or signal generating device is, of course, thus advantageously designed in such a way that the device is directly and easily accessible for an operator.

According to a preferred embodiment, the treatment apparatus comprises two cover parts, with one cover part being attached in a pivotable manner at each free end of the bearing part. On the one hand, an exact adjustment of the treatment apparatus to the head of the patient is thereby made possible and on the other hand, the patient can easily place his head in the treatment apparatus. In contrast to when only one cover part is provided, the pivoting angle for each cover part, when two cover parts are provided, must not be as great as is required in the case of one cover part, which again simplifies the attachment of signal transmission devices between the bearing part and the cover parts. More than two cover parts can, of course, also be provided, with, in this case, each further cover part being pivotably attached in a similar manner to corresponding other cover parts. The signal transmission devices must also be provided between the corresponding cover parts in this case.

In the closed state, the cover parts and the bearing part advantageously complete to form a ring. The patient's head can thereby be surrounded by an almost closed ring so that a specific treatment is possible. Even if the patient turns his head in the treatment apparatus, the alignment of the treatment on the head is guaranteed.

A plate-like element having two main sides is preferably used as the pivoting device, with a rotating means for a relative rotation between the bearing part and the plate-like element and a rotating means for a relative rotation between the plate-like element and the cover part being provided. The plate-like element is attached to the bearing part in such a way that one of its main surfaces abuts one of the ends of the plate-like element. A pivoting between the bearing part and the cover parts is made possible by this simple and reliable pivoting device which can be realised as a rotating device, for example, by means of bolts, whilst at the same time keeping a good adjustment of the parts in relation to one another and to the head of the patient.

The treatment apparatus is thereby advantageously designed in such a way that the pivoting device can be operated by hand but, however, has such a frictional resistance in itself that it remains in the preset position without a force effect. Therefore, no additional devices or tools are necessary for adjusting the treatment apparatus to the head of the patient.

The bearing part is advantageously attached to one of the main surfaces of the plate-like element, whereas the cover part or parts are provided on the other main surface of the plate-like element. This again serves a specific arrangement of the treatment apparatus around the head of the patient.

This arrangement is particularly advantageous if the treatment apparatus is a magnetic field therapy device. By means of a magnetic field therapy device, fields having specific signals such as pulsating fields which are applied by a magnetic field, can be specifically guided onto the patient's head. This has proven successful for the treatment of tinnitus in particular.

### BRIEF DESCRIPTION OF THE DRAWINGS

The invention is described in more detail below purely as an example by means of the figures in which:

FIG. 1 shows a perspective view of a treatment bed according to the invention;

FIG. 2 shows a rear perspective view of an embodiment of the treatment bed according to the invention;

FIG. 3 shows a front view of an embodiment of the treatment bed according to the invention;

FIG. 4 shows a perspective view of a treatment apparatus according to an embodiment of the invention, in which the treatment apparatus is attached to a bed according to the invention; and

FIG. 5 shows a top view of a treatment apparatus according to the invention in the integrated state in a treatment bed according to the invention.

### DETAILED DESCRIPTION OF THE INVENTION

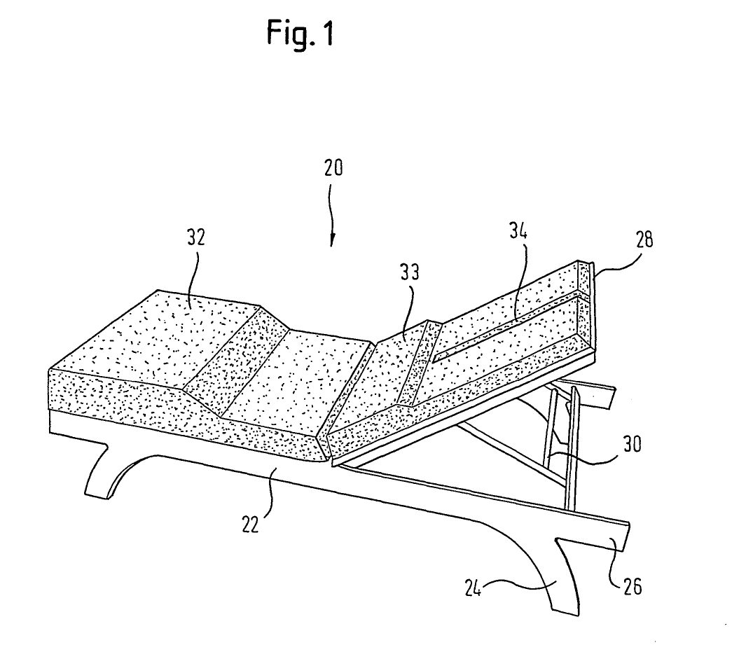

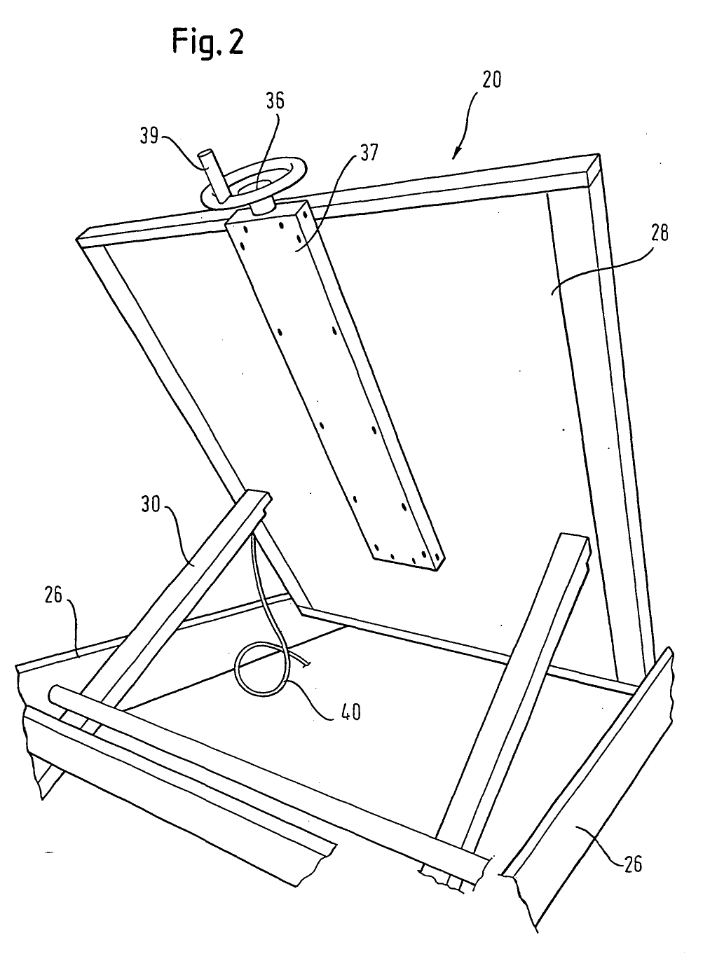

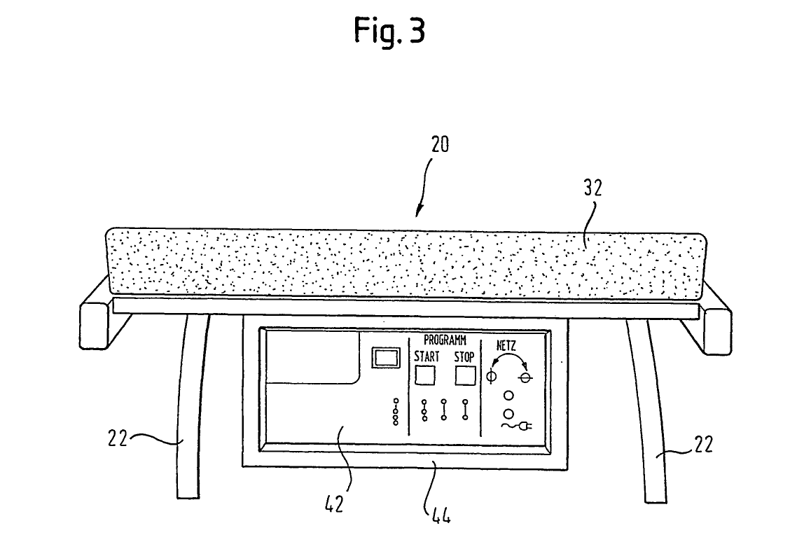

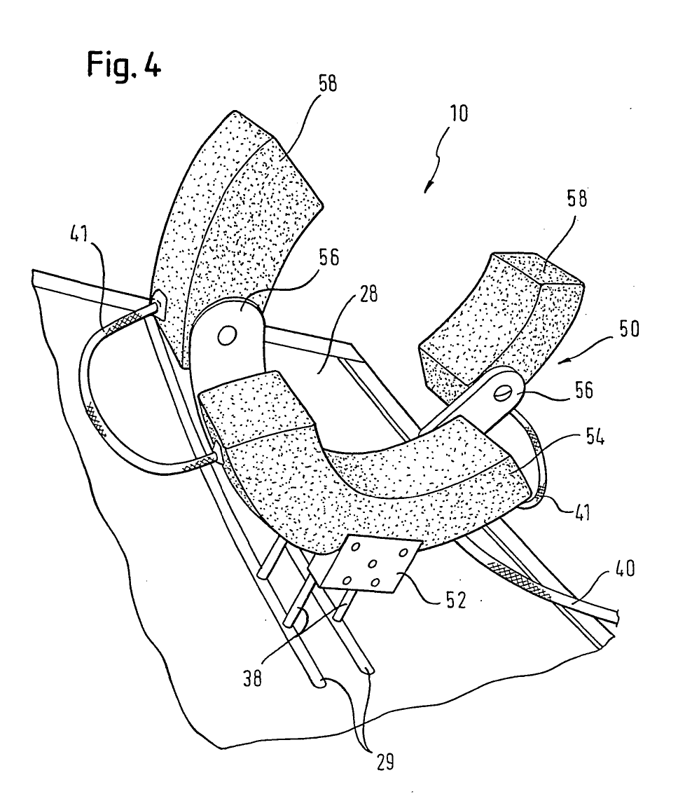

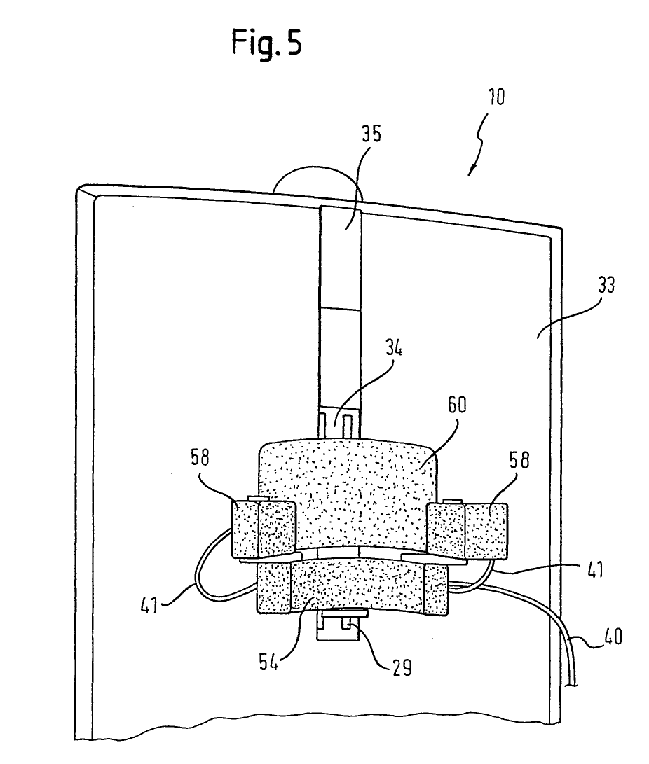

FIG. 1 shows a perspective view of a treatment bed 20 according to the invention which forms part of a treatment device 10 according to the invention as is shown in part in FIGS. 4 and 5 for example. FIGS. 2 and 3 respectively show a rear perspective view of the treatment bed according to the invention and a front view of the treatment bed.

It can be seen in FIG. 1 that the treatment bed 20 basically comprises a frame 22 which is provided with feet 24. With the feet 24, the treatment bed stands on the floor in approximately the shape of a normal bed. The height of the treatment bed 20 is advantageously selected in such a way that a patient can easily sit himself on the treatment bed having an attached bearing element. The measurements in the longitudinal direction and widthwise direction of the treatment bed 20 therefore correspond to the measurements of a normal bed, however the measurements should always be measured in such a way that a patient can rest comfortably on the treatment bed 20.

In addition to the basically horizontally extending supporting body 26 with the feet 24 attached thereto, the frame 22 also comprises a head part 28. This head part is inclined from the horizontal, i.e. there is an angle of greater than 0° between the basically horizontally extending supporting body 26 of the frame 22 and the head part 28 of the frame 22. Depending on whether or not the frame of the treatment bed has an adjustment mechanism for inclining the head part 28, this angle can be adjusted to the patient's wishes within a certain range. A support part 30 is also provided in the embodiment shown in FIG. 1, which can engage in the horizontally extending supporting body in different positions. This support part 30 is attached to the head part 28 via a hinge. A desired inclination of the head part 28 of the frame 22 can thus be set by means of a different engaging of the support part 30 in the supporting body 26 of the frame 22. It is, of course, alternatively possible to design the treatment bed in such a way that the inclination of the head part is fixedly predetermined and cannot be altered. The bearing element surface of the frame of the treatment bed can basically be freely designed. For example, a plate-like, continuous support for the bearing element or bearing elements 32, 33 of the treatment bed can be provided. Alternatively struts, i.e. no continuous plate, can also be provided, which support the bearing elements 32, 33.

The material from which the frame of the treatment bed is made can also be almost freely selected. Wood offers, for example, an optically appealing appearance. If the treatment bed 20 is used to treat tinnitus, for example, no significant dirtying can be expected and the hygiene requirements are also not notably increased, and thus wood would be suitable in this case. It is, of course, alternatively possible to construct the frame 22 of the treatment bed from another material, for example metal.

The treatment bed 20 furthermore comprises bearing elements 32, 33. In the embodiment shown in FIG. 1, these are padded bearing elements which, for example, consist of a foam body with a cover. It is desirable for the comfort of the patient if the bearing elements 32, 33 are designed to give to a certain extent.

As can clearly be seen from FIG. 1, the bearing elements 32, 33 are ergonomically shaped. This particularly means that the bearing element 32 placed in the leg area of the patient has a depression for the patient's buttocks. In the embodiment shown in FIG. 1, this depression remains unchanged over the widthwise direction of the bearing element. However, the depression can, of course, also be variably designed in the widthwise direction if this appears necessary. The depression for the buttocks in contrast to the elevated position for the legs ensures that the patient's legs are resting in an elevated position during the treatment, which benefits the well-being of the patient owing to a favourable influence on the blood circulation.

The bearing element 33 provided in the head and torso area of a patient abuts the bearing element 32 at the same height so that no bulge or the like exists in the transition region. To avoid bulges, it is also advantageous to design the bearing elements 32, 33 in two parts, in particular if there is an inclined head part 28 of the treatment bed since otherwise bulges could be caused in the transition region. The bearing element 33 provided in the head/torso area furthermore also comprises a depression up to the head area as compared to the area supporting the lumbar region. This depression serves in particular to quickly signal to the patient that he is slipping out of the correct treatment position. It additionally facilitates the placement of the treatment apparatus which is guided via supports in the recesses provided in the frame 22 and the bearing element 33.

As can be seen from FIG. 5, a recess 34 is formed in the bearing element 33 to guide and ensure the shifting of the treatment apparatus. This recess 34 exposes the elongated recesses 29 extending in the longitudinal direction of the frame and in particular the head part 28 thereof, which are provided in duplicate in this embodiment, through which the supports for the treatment apparatus are guided (see. FIG. 4; FIG. 4 is shown without the bearing element 33). It can furthermore be seen from FIG. 5 that the recess 34 in the bearing element 33 can be covered at least partially by inserts 35. These inserts help in particular to ensure an appealing appearance.

FIG. 2 shows a rear perspective view of the treatment bed according to the invention. In addition to the elements already discussed in connection with FIG. 1, an adjustment mechanism 36 for the support 38 (see FIG. 4) of a treatment apparatus can also be clearly recognised in this figure. The adjustment mechanism is fixed on the rear side of the head part 28, i.e. on the side facing away from the bearing element side, by means of screws or the like. In particular, a plate 37 is fixed via the recess 34 in the head part 28 of the frame 22, which, for its part, contains the adjustment mechanism for the supports 38, for example in the form of a spindle, said adjustment mechanism being operable via a crank 39. The plate-like element 37 is arranged in such a way that the supports 38 run in the recesses 29 and 34 provided therefor in the frame and the bearing element 33. Other shifting mechanisms for the supports 38 are, of course, also possible. The fixing of the plate 37, which is carried out in the shown embodiment to the head part 28 by means of screws, can, of course, also be designed in another manner.

Furthermore, a cable 40 can also be seen in FIG. 2, which extends from a treatment apparatus to a signal generating device and/or control device. This cable 40 is preferably guided on the underside of the treatment bed so that there is no danger of the operator or patient tripping.

FIG. 3 shows a front view of the treatment bed according to the invention. It can be seen that a control device and/or a signal generating device 42 is placed in a frame-like receptacle 44 provided therefor on the treatment apparatus. The receptacle 44 is adapted to the size of the signal generating device and the control device. It is furthermore attached in such a way that an operator can reach it without any problems. Attachment is, of course, not only limited to the shown position on the frame 22. Any position on the frame 22 can be selected provided that it remains easily accessible.

FIGS. 4 and 5 show a treatment device 10 which comprises a treatment apparatus 50 in addition to the treatment bed described in connection with FIGS. 1 to 3. The treatment apparatus 50 is provided via a fixing device 52, which, in the shown case, is a simple screw connection and a plate, with the supports 38 which form the connection to the adjustment mechanism in the longitudinal direction along the treatment bed. Four supports 38 are provided in the shown embodiment. It is, of course, possible to provide more than four or less than four such supports provided that the support and shifting function remains ensured by the adjustment mechanism. Different possibilities to the one shown are also conceivable for the fixing device 52 of the treatment apparatus 50 on the supports 38.

The treatment apparatus 50 in the shown embodiment furthermore comprises a bearing part 54 which is shaped as a ring segment. The ring segment is arranged in such a way that it basically takes the shape of the neck of a patient. The ring segment 54 is advantageously made of a flexible bearing element, and thus the head or neck of the patient rests softly when it is lying in the bearing element. Plate-like elements 56 are provided at the free ends of the bearing part 54, which form the connection to the cover parts 58 which are also designed in a soft manner to avoid injuries. The connections between the bearing part 54 and the plate-like elements 56 and between the plate-like elements 56 and the cover parts 58, of which two are provided in the shown embodiment, are respectively carried out in such a manner that the plate-like element 56 can be pivoted against the respective other element. This means that in the closed state, the bearing part 54 and the cover parts 58 form an essentially ring-shaped configuration having only very small clearances or no clearances at all. The plate-like elements 56 are advantageously provided in such a way that the desired pivoted positions between the bearing part 54 and the cover part 58 can be set by hand. Nevertheless, the set positions of these parts should not change without an external force effect owing to the friction between the plate-like elements 56 and the bearing part 54 or the cover parts 58, so that the treatment apparatus remains in a stable position.

It can furthermore be clearly seen from FIG. 4 that a signal-transmitting connection is provided by means of the cable 40, first from an external control unit and/or signal generating unit to the treatment apparatus 50 and then within the treatment apparatus via the cable 41 between the bearing part 54 and the cover parts 58.

So that the whole of the patient's head rests comfortably during the treatment, additional bearing elements, such as, for example, the bearing element 60 as shown in FIG. 5, can be provided.

All parts coming into direct contact with the patient's body are preferably designed to give, e.g. they are made of a foam material for example.

The essential aspect of the invention is to construct a treatment device, for example, for the treatment of tinnitus using magnetic field therapy, which can be adapted to individual needs and which basically includes a treatment bed and a treatment apparatus.

1. A treatment bed (20) comprising
a frame (22), and
at least one bearing element (32, 33) which can be placed on the frame (22), wherein at least one elongated recess (29, 34) extending in the longitudinal direction of the frame is provided in the frame and the bearing element, which extends from a head section of the treatment bed (20) into a central area in the widthwise direction.

2. A treatment bed (20) according to claim 1, characterised in that the frame (22) comprises an inclinable head section.

3. A treatment bed (20) according to claim 2, characterised in that the inclinable head section takes up approximately half of the length of the treatment bed.

4. A treatment bed (20) according to one of claims 2 or 3, characterised in that the frame comprises a supporting body (26) which extends basically horizontally, a head part (28) which is pivotably attached in the central area of the supporting body in the longitudinal direction, and a supporting part (30) attached to the head part by means of a hinge, which can be engaged with its free end in an engagement area provided on the supporting body (26) in order to cause an inclination of the head part (28).

5. A treatment bed (20) according to claim 4, characterised in that several engagement areas are provided on the supporting body (26) so that the head part can assume different inclinations.

6. A treatment bed (20) according to one of the preceding claims, characterised in that the frame (22) is made of wood.

7. A treatment bed (20) according to one of claims 1 to 5, characterised in that the frame (22) is made of metal.

8. A treatment bed (20) according to one of the preceding claims, characterised in that the at least one bearing element (32, 33) is a softly padded bearing element.

9. A treatment bed (20) according to one of the preceding claims, characterised in that the bearing element comprises a separate head part (33) and foot part (32).

10. A treatment bed (20) according to one of the preceding claims, characterised in that the bearing element (32) has a depression in the area in which the buttocks of a patient rest, whereas the area in which the legs rest is raised.

11. A treatment bed (20) according to one of the preceding claims, characterised in that the bearing element (32) comprises a recess (34) in the area of the at least one recess in the frame, which exposes at least the excess (29) provided in the frame, and in that at least one filling piece (35) is provided for the recess in the bearing element in order to cover the recess (34) at least partially.

12. A treatment bed (20) according to one of the preceding claims, characterised in that the bearing element (33) is designed so as to be depressed in the area of the recess (34) as compared to a raised area for supporting the patient's lumbar region.

13. A treatment bed (20) according to one of the preceding claims, further comprising an adjustment mechanism (36) for adjusting a treatment apparatus (50) for the head area of a patient in the longitudinal direction.

14. A treatment bed (20) according to claim 13, characterised in that the adjustment mechanism (36) comprises a plate (37) fixed on the side facing away from the bearing element side, to which at least one support (38) for the treatment apparatus is attached in a shiftable manner.

15. A treatment bed (20) according to claim 14, characterised in that the at least one support (38) is shiftable by means of a spindle and a crank (39) for operating the spindle.

16. A treatment bed (20) according to one of the preceding claims, characterised in that a receptacle (44) for a signal generating device and/or control device (42) for a treatment apparatus is provided.

17. A treatment apparatus (50) comprising
a bearing part for the head of a patient, said bearing part essentially taking the shape of a ring segment;
at least one cover part placed on at least one free end of the bearing part by means of a pivoting device (56), with the at least one cover part (58) being connected to the bearing part by means of a signal transmission device (41);
a fixing device (52) for the treatment apparatus; and
a signal generating device and/or control device (42) connected to the treatment apparatus.

18. A treatment apparatus (50) according to claim 17, characterised in that two cover parts (58) are provided, with one cover part (58) being pivotably attached to each free end of the bearing part (54).

19. A treatment apparatus (50) according to claim 17 or 18, characterised in that the bearing part (54) and the at least one cover part (58) complete to form a ring.

20. A treatment apparatus (50) according to one of claims 17 to 19, characterised in that pivoting device comprises a plate-like element (56) on which a rotating means for a relative rotation between the bearing part (54) and the plate-like element (56) and a rotating means between the plate-like element (56) and the cover part (58) is provided.

21. A treatment apparatus (50) according to claim 20, characterised in that the bearing part (54) is provided on a main surface of the plate-like element (56) and the at least one cover part (58) is provided on the other main surface of the plate-like element (56).

22. A treatment apparatus (50) according to one of claims 17 to 21, characterised in that said treatment apparatus is a magnetic field therapy device.

23. A treatment device (10) comprising a treatment bed (20) according to one of claims 1 to 16 and a treatment apparatus (50) according to one of claims 17 to 22.
